(19) United States
(12) Patent Application Publication
(10) Pub. No.: US 2008/0039528 A1
(43) Pub. Date: Feb. 14, 2008

(54) Zinc finger ejectors and methods of use thereof

(75) Inventor: Duy H. Hua, Manhattan, KS (US)

Correspondence Address: HOVEY WILLIAMS LLP, 2405 GRAND BLVD., SUITE 400, KANSAS CITY, MO, 64108, US

(73) Assignee: KANSAS STATE UNIVERSITY RESEARCH FOUNDATION

(21) Appl. No.: 11/081,202

(22) Filed: Mar. 16, 2005

Related U.S. Application Data
(60) Provisional application No. 60/553,882, filed on Mar. 17, 2004.

(51) Int. Cl. C12N 7/06; A61K 31/19; C07K 1/14

(52) U.S. Cl. 514/575; 435/238; 530/344

(57) ABSTRACT

Techniques for the chelation or ejection of Zn2+ from zinc finger peptides are disclosed, which can be useful in the treatment or control of viruses and viral diseases and malaria. The invention comprises contacting a zinc finger peptide with an effective amount of a bishydroxamic acid or salt thereof, either in vivo or in vitro.

### CROSS REFERENCE TO RELATED APPLICATION

This application claims the benefit of Provisional Application No. 60/553,882, filed Mar. 17, 2004. This Provisional Application is incorporated by reference herein.

### SEQUENCE LISTING

A printed Sequence Listing accompanies this application, and has also been submitted with identical contents in the form of a computer-readable ASCII file on a floppy diskette and a CDROM. Contents of this Sequence Listing are incorporated by reference.

### BACKGROUND OF THE INVENTION

1. Field of the Invention

The present invention is broadly concerned with methods for the chelation and ejection of zinc from zinc finger peptides such as those involved in viral replication. More particularly, the invention is concerned with such methods, as well as certain novel compounds, making use of bishydroxamic acids and their synthetic precursors such as diesters and dicarboxylic acids.

2. Description of the Prior Art

The zinc-finger motifs (sometimes classified as C2H2, C4 or C6 motifs) of proteins are required for efficient reverse transcription, initial integration processes, protection of newly synthesized viral DNA, virus uncoating as well as transcription inhibition and m-RNA regulation. Hence, removal of zinc ions from zinc-finger viral proteins or viral RNA polymerases leads to the inhibition of viral replication in vitro and in vivo. A recent review by Kesel (Biiorg. & Med. Chem. 2003, 11, 4599-4613) depicted the zinc finger I and II motif in HIV-1 and existence of similar motifs in various viruses such as polio, human coxsackie, SARS, rabies, human parainfluenza, measles, human respiratory syncytial, human hepatitis, Dengue, West Nile, Ebola, to name a few. The use of zinc ejectors as antiviral agents is still in its infancy and careful studies of selective zinc chelators may provide safe and less expensive antiviral drugs.

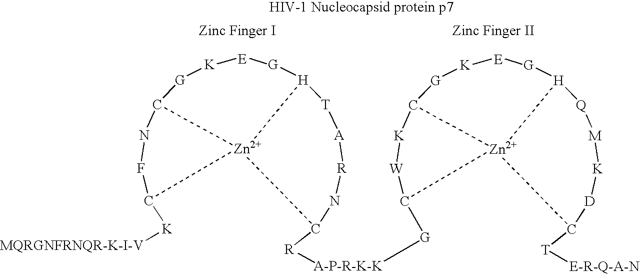

### SUMMARY OF THE INVENTION

The present invention is broadly concerned with techniques for chelating or ejecting Zn2+ from a zinc finger peptide which may be a part of viral proteins or viral RNA polymerases, for example, so as to inhibit viral replication. Broadly speaking, the methods of the invention involve contacting the peptide with an effective amount of a bishydroxamic acid or salt thereof, and thereby causing the desired chelation or ejection. The methods may be conducted in vivo or in vitro. Preferred bishydroxamic acids are symmetrical, and are selected from the group consisting of compounds 2, 3, 15A-15D, inclusive, and 16, described below. The bishydroxamic acids may be readily prepared by the alkylation of methyl 4-hydroxybenzoate with various dihalo-alkane, -alkene, and -ether followed by reaction with hydroxylamine.

The methods of the invention find utility in the control or treatment of a variety of viruses and viral diseases, such as HIV, polio, human coxsackie, SARS, rabies, human parainfluenza, measles, human respiratory syncytial, human hepatitis, Dengue, West Nile and Ebola. Bioactivities of selected bishydroxamic acids have been evaluated against malarial Plasmodium falciparum and Leishmania donovani parasites.

### DETAILED DESCRIPTION OF THE PREFERRED EMBODIMENTS

A class of symmetrical bishydroxamic acids were prepared, such as compounds 2 and 3 depicted in Scheme 1 below. For example, alkylation of 2 eq. of methyl 4-hydroxybenzoate (4) with K2CO3 in acetone and 1 eq. of di(2-bromoethyl)ether and 1,4-E-dichloro-2-butene, separately followed by basic amidation with hydroxylamine gave 3 and 2, respectively in good yields. Due to the trans double bond functionality of 2, presumably, 2 acts as monodentate, in which folding of the molecule to adapt a similar structure as that of 15A will create an energetically unfavorable structure. Hence, chelation with Zn2+ would require two molecules of 2; while only one molecule of bishydroxamic acid 3 is required for chelation with Zn2+ (such as the structure 15A in Scheme 2). Thus the activity of 3 is twice of that of 2. Zn2+ is known to form tetracoordinate complex with oxygen atoms.

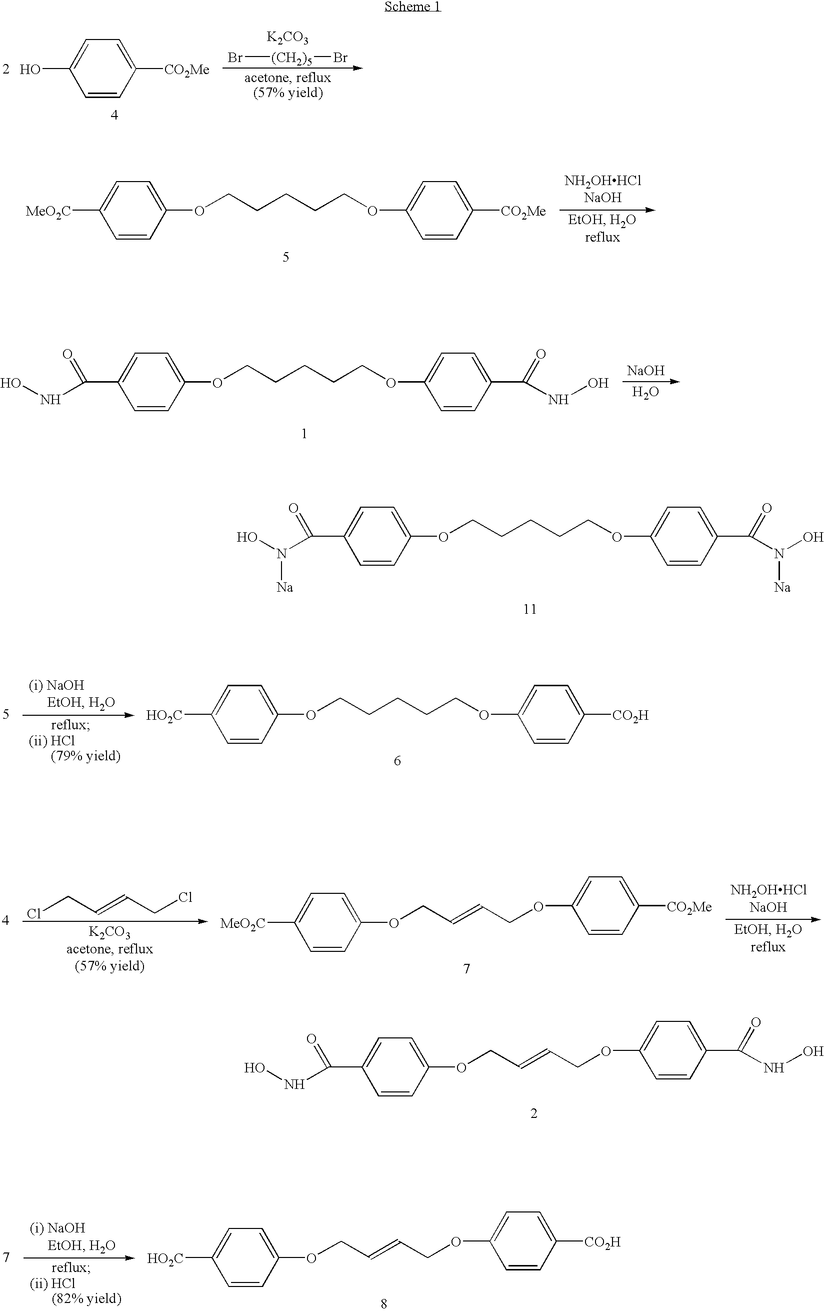

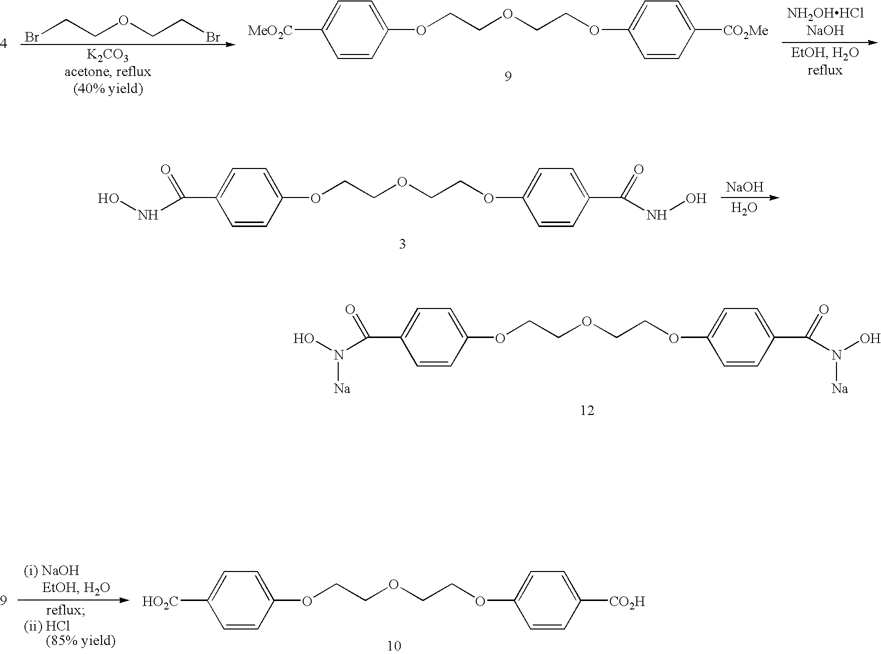

Two classes of cyclophane-based bishydroxamic acids are also synthesized (see Scheme 2 below). Compounds 15A-15D mimic 3 (see Scheme 1), and rigid bishydroxamic acids 16 (n=3, 4, 5) contain symmetrical bishydroxamic moieties providing a best fit with zinc²⁺ ion. The syntheses of compounds 15A-15D and 16 are depicted in Scheme 2. Alkylation of disodium salt of methyl 3,5-(dihydroxymethyl)benzoate (18A) (derived from the corresponding diol with 2 eq. of NaH) with methyl 3,5-(dibromomethyl)benzoate (17) in THF produces cyclophane 19A. Methyl 3,5-(dihydroxymethyl)benzoate (18) obtains from basic hydrolysis of dibromide 17 with H₂O-THF-NaHCO₃. Similar Williamson alkylations of thiolate 18B and amide 18C with 17 gives sulfide 19B and amide 19C, respectively. Thiolate 18B and amide 18C obtain from the alkylations of 17 with H₂S-THF-NaHCO₃ and 2 eq. of H₂NCO₂Me-THF-NaH, respectively. Treatment of 19A-19C with NaOH—H₂NOH in ethanol-water¹ produces bishydroxamic acids 15A-15C. The carbamate function (NCO₂Me) of 19C is stable under the basic hydroxylamine reaction conditions. Bishydroxamic acid 15D contains two amino acid functions as the linkers, and it is synthesized from the alkylation of 2 eq. of protected glycine 20 with 2 eq. of NaH and dibromide 17 in THF to give ester 21. Cyclophane formation of 21 with 2 eq. of NaH and 17 in THF gives 22. Basic hydroxyamidation of 22 with NaOH—H₂NOH followed by removal of the protecting groups with HCl in THF—H₂O give bishydroxamic acid 15D. It should be noted that the length of the two side arms (CH₂WCH₂) of 15A-15D can be changed to longer alkyl chains if necessary.

Rigid Bishydroxamic acids 16 (n=3, 4, 5) are synthesized from the alkylation of anion 18A with dibromide 23 [derived from the dibromination of 3,5-dimethyl-1-(t-butyldimethylsilyloxy)benzene with NBS and benzoyl peroxide⁷] in THF. Transformation of cyclophane 24 to 16 is carried out by the sequence: (i) removal of the silyl ether protecting group with n-Bu₄NF in THF; (ii) alkylation with NaH and 0.5 eq. of 1,3-dibromopropane (n=3), 1,4-dibromobutane (n=4), and 1,5-dibromopentane (n=5); and (iii) amidation with NaOH—H₂NOH in ethanol-water. Similar to that described in 15, the length of the side chains (CH₂OCH₂) of 16 can be lengthened if necessary. The syntheses provide a range of rigid and selective zinc chelators, which may serve to eject zinc ion from viral RNA zinc-finger motifs.

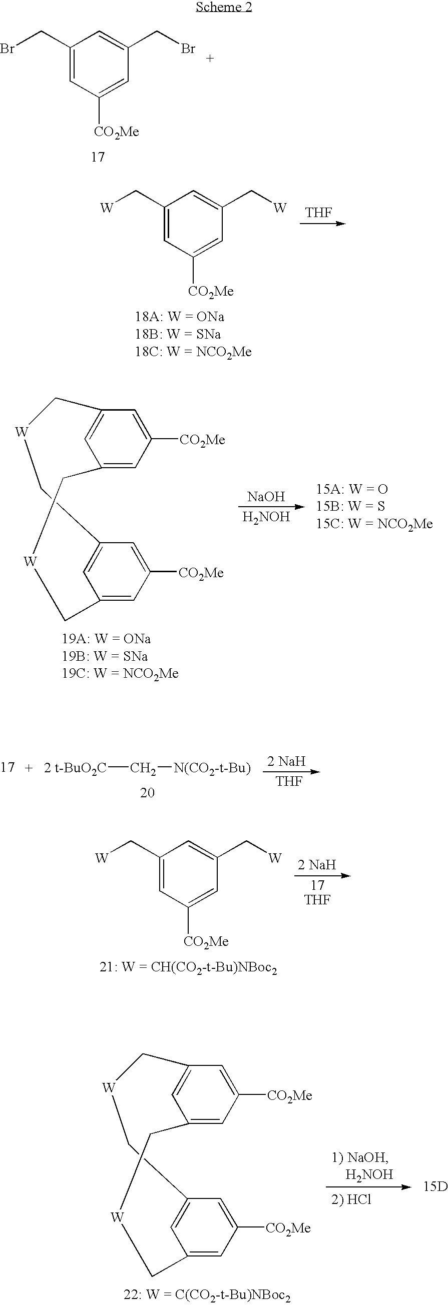

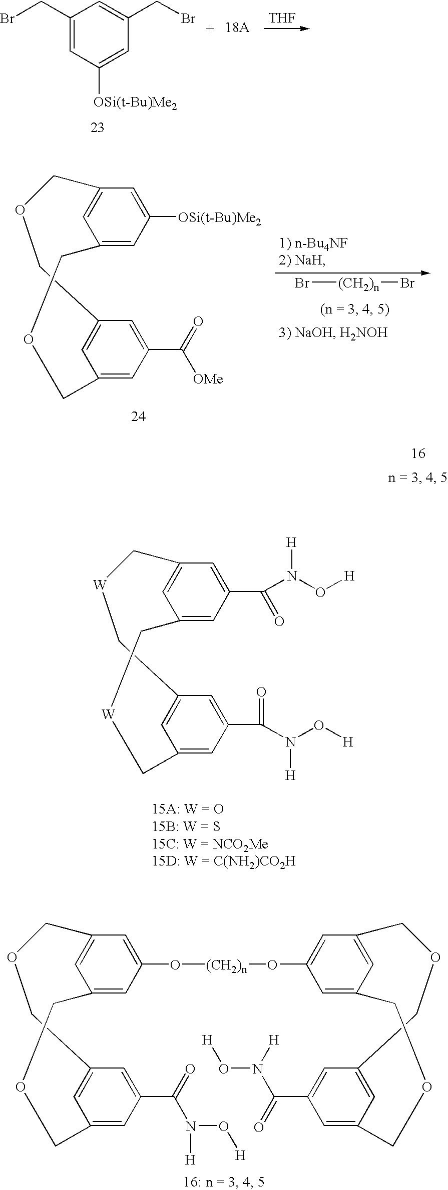

### Example I

### Syntheses of Compounds According to Scheme 1

Owing to the chelation ability of bishydroxamic acids with metals, three symmetrical bishydroxamic acids 1-3, along with their corresponding sodium salts, methyl esters, and carboxylic acids were synthesized. As shown in Scheme 1, the syntheses of the methyl esters, carboxylic acids, and hydroxamic acids and their sodium salts were carried out via a typical alkylation of the hydroxy function of methyl 4-hydroxybenzoate (4) followed by either reaction with hydroxylamine [9] to provide bishydroxamic acids or with base to give biscarboxylic acids. Hence, treatment of 4 with potassium carbonate and 1,5-dibromopentane, trans-1,4-dichloro-2-butene, and bis-2-bromoethyl ether separately in refluxing acetone provided methyl esters 5 (57% yield), 7 (57% yield), and 9 (40% yield), respectively. Heating of esters 5, [10] 7, and 9 [11] with hydroxylamine and sodium hydroxide in refluxing ethanol-water produced bishydroxamic acids 1, [4] 2, and 3, respectively, in good yields. Compounds 2 and 3 have not been reported previously. Carboxylic acids 6, 8, and 10 were readily obtained from the basic hydrolysis of esters 5, 7, and 9, respectively, with sodium hydroxide in refluxing ethanol and water, followed by acidification, in 79, 82, and 85% yield, respectively. Sodium salts 11 and 12 were obtained by treating bishydroxamic acids 1 and 3, respectively, with sodium hydroxide. Carboxylic acids 6, [12] 8, [13] and 10 [12 and 14] are known compounds, and usages in materials have been reported.

Nuclear magnetic resonance spectra were obtained at either 400 or 200 MHz for ¹H and either 100 or 50 MHz for ¹³C, and reported in ppm. Mass spectra were taken from an IonSpec HiResMALDI mass spectrometer using 2,5-dihydroxybenzoic acid as a matrix.

The following set forth details of preferred synthetic techniques.

1,5-Bis-(4-methoxycarbonylphenoxy)pentane (5). A mixture of 11.7 g (0.077 mol) of methyl 4-hydroxybenzoate (4), 48.0 g (0.347 mol) of K₂CO₃, and 8.0 g (0.035 mol) of 1,5-dibromopentane in 100 mL of acetone was stirred under reflux for 31 h under argon. After the mixture was cooled to room temperature, the inorganic salts were removed by filtration, and the filtrate was concentrated on a rotary evaporator to remove most of the acetone. The residue was dissolved in 100 mL of dichloromethane, washed with aqueous NaOH (30 mL; 0.5 M solution), dried (MgSO₄), and concentrated to dryness to give 7.37 g (57% yield) of 5 [10] as a white solid; mp 93-95° C. Ester 5 was crystallized from ethyl acetate to give white crystals. ¹H NMR (CDCl₃) 7.98 (d, J=8.4 Hz, 4H, Ar), 6.90 (d, J=8.4 Hz, 4H, Ar), 4.05 (t, J=6.4 Hz, 4H, OCH₂), 3.88 (s, 6H, OCH₃), 1.98-1.60 (m, 6H, CH₂); ¹³C NMR (CDCl₃) 166.8, 162.7, 131.5, 122.4, 114.0, 67.8, 51.8, 28.8, 22.6.

trans-1,4-Bis-(4-methoxycarbonylphenoxy)-2-butene (7). By a procedure similar to that above, 3.30 g (26.4 mmol) of trans-1,4-dichloro-2-butene, 8.85 g (58.2 mmol) of 4, 36.5 g (0.264 mol) of K₂CO₃, and 40 mL of acetone gave 3.75 g (40% yield) of 7 as a white solid after crystallization from ethyl acetate. The mother liquor from crystallization was concentrated and column chromatographed on silica gel to give 1.65 g (17% yield; a total of 57% yield) of 7. Mp 153-155° C.; ¹H NMR (CDCl₃) 7.98 (d, J=7 Hz, 4H, Ar), 6.92 (d, J=7 Hz, 4H, Ar), 6.10 (t, J=1.5 Hz, 2H, ═CH), 4.64 (d, J=1.5 Hz, 4H, CH₂), 3.88 (s, 6H, OCH₃); ¹³C NMR (CDCl₃) 166.7, 162.1, 131.6, 128.0, 122.9, 114.2, 67.6, 51.8. HRMS calcd for C₂₀H₂₀O₆Na: 379.1158, found 379.1154.

2,2′-Bis-(4-methoxycarbonylphenoxy)ethyl ether (9). By a procedure similar to that above, 2.00 g (7.8 mmol) of 2,2′-di(bromoethyl)ether, 2.68 g (17.6 mmol) of 4, 11.0 g (79.6 mmol) of K₂CO₃, and 15 mL of acetone gave 2.47 g (85% yield) of 9 [11] as a white solid after crystallization from ethyl acetate. Mp 120-123° C.; ¹H NMR (CDCl₃) 7.97 (d, J=8.5 Hz, 4H, Ar), 6.92 (d, J=8.5 Hz, 4H, Ar), 4.21 (t, J=5 Hz, 4H, CH₂), 3.95 (t, J=5 Hz, 4H, CH₂), 3.88 (s, 6H, OCH₃); ¹³C NMR (CDCl₃) 166.7, 162.4, 131.5, 122.8, 114.1, 69.7, 67.5, 51.8.

1,5-Bis-[4-(hydroxyaminocarbonyl)phenoxy]pentane (1). To a mixture of 0.9 g (2.4 mmol) of ester 5 in 24 mL of 0.5 M solution of NH₂OH.HCl (12 mmol) was added 4.8 mL (28.8 mmol) of 6 M aqueous NaOH solution. The mixture was refluxed for 5 min, then cooled to room temperature, and 28.8 mL (28.8 mmol) of 1 N HCl was added. The precipitated solids, was collected by filtration and dried thoroughly under vacuum, to weight 0.74 g (82% crude yield). ¹H NMR spectrum indicated the presence of 1 and a small amount of biscarboxylic acid 6. Recrystallization from DMSO-water (10:1) three times gave 1, [4] 0.51 g (57% yield): mp 200-202° C. (lit. [4] 202-203° C.); ¹H NMR (DMSO-d₆) 7.69 (d, J=8.8 Hz, 4H, Ar), 6.96 (d, J=8.8 Hz, 4H, Ar), 4.03 (t, J=6.4 Hz, 4H, OCH₂), 1.78 (m, 4H, CH₂), 1.55 (m, 2H, CH₂); ¹³C NMR (DMSO-d₆) 165.6, 156.7, 131.2, 128.5, 114.1, 67.6, 28.1, 22.0.

trans-1,4-Bis[4-(hydroxyaminocarbonyl)phenoxy]-2-butene (2). A mixture of 0.45 g (1.26 mmol) of ester 7, 12 mL of 0.5 M solution of NH₂OH.HCl (6 mmol), and 1.0 mL (6 mmol) of 6 M aqueous NaOH was treated similarly to that described above for the preparation of 1. Three crystallizations of the crude product from DMSO-water (10:1) gave 2, 0.18 g (40% yield): mp 205-210° C.; ¹H NMR (DMSO-d₆) 7.70 (d, J=8.8 Hz, 4H, Ar), 6.99 (d, J=8.8 Hz, 4H, Ar), 6.07 (bs, 2H, ═CH), 4.62 (bs, 4H, OCH₂); ¹³C NMR (DMSO-d₆) 166.9, 160.4, 131.3, 128.6, 125.2, 114.3, 67.3. HRMS calcd for C₁₈H₈N₂O₆Na: 381.1063, found 381.1064.

2,2′-Bis-[(4-hydroxyaminocarbonyl)phenoxy]ethyl ether (3). A mixture of 1.0 g (2.67 mmol) of ester 9, 27 mL of 0.5 M solution of NH₂OH.HCl (13.5 mmol), and 5.3 mL (32 mmol) of 6 M aqueous NaOH was treated similarly to that described above for the preparation of 1. Two crystallizations of the crude product from DMSO-water (10:1) gave 2, 0.804 g (80% yield): mp 220-226° C.; ¹H NMR (DMSO-d₆) 7.68 (d, J=8 Hz, 4H, Ar), 6.97 (d, J=8 Hz, 4H, Ar), 4.17 (t, J=4 Hz, 4H, OCH₂), 3.80 (t, J=4 Hz, 4H, OCH₂); ¹³C NMR (DMSO-d₆) 164.2, 160.8, 128.8, 125.1, 114.2, 69.0, 67.5. HRMS calcd for C₁₈H₂₀N₂O₇Na: 399.1168, found 399.1155.

1,5-Bis-[4-(hydroxycarbonyl)phenoxy]pentane (6). To a solution of 0.30 g (0.81 mmol) of ester 5 in 20 mL of ethanol, 1.6 mL of 6 M aqueous NaOH was added and the solution was stirred under reflux for 10 min. After being cooled to room temperature, the solution was acidified with 1 N HCl to pH 1, and the precipitated white solids were collected by filtration, washed with water, dried under vacuum, and crystallized from ethanol to give 0.22 g (79% yield) of 6: [12] mp 283-286° C.; ¹H NMR (DMSO-d₆) 7.86 (d, J=8.4 Hz, 4H, Ar), 6.99 (d, J=8.4 Hz, 4H, Ar), 4.06 (t, J=7 Hz, 4H, OCH₂), 1.79 (pent, J=7 Hz, 4H, CH₂), 1.56 (pent, J=7 Hz, 2H, CH₂); ¹³C NMR (DMSO-d₆) 164.0, 160.8, 124.7, 122.8, 114.0, 67.5, 28.2, 22.0.

trans-1,4-Bis-[4-(hydroxycarbonyl)phenoxy]-2-butene (8). A mixture of 0.10 g (0.28 mmol) of ester 7, 3.0 mL of ethanol, and 2.0 mL (12 mmol) of 6 N NaOH was treated similarly to that described above for the preparation of 6 to give 67 mg (74% yield) of 8: [13] mp>290° C.; ¹H NMR (DMSO-d₆) 7.88 (d, J=9 Hz, 4H, Ar), 7.03 (d, J=9 Hz, 4H, Ar), 6.09 (bs, 2H, ═CH), 4.69 (bs, 4H, CH₂O); ¹³C NMR (DMSO-d₆) 166.9, 161.7, 131.3, 128.2, 123.2, 114.5, 67.4.

2,2′-Bis-[(4-hydroxycarbonyl)phenoxy]ethyl ether (10). A mixture of 0.15 g (0.40 mmol) of 9, 5.0 mL of ethanol, and 2.7 mL (16.2 mmol) of 6 N NaOH was treated similarly to that described above for the preparation of 6 to give 0.115 g (83% yield) of 10: [12 and 14] mp 308-310° C.; ¹H NMR (DMSO-d₆) 7.86 (d, J=7.5 Hz, 4H, Ar), 7.00 (d, J=7.5 Hz, 4H, Ar), 4.18 (t, J=3.5 Hz, 4H, OCH₂), 3.83 (t, J=3.5 Hz, 4H, OCH₂); ¹³C NMR (DMSO-d₆) 166.9, 162.0, 131.3, 123.0, 114.3, 68.9, 67.4.

1,5-Bis-[4-(hydroxy-sodio-aminocarbonyl)phenoxy]pentane (11). To a solution of 0.10 g (0.27 mmol) of 1 in 1 mL of water was added 21.4 mg (0.53 mmol) of NaOH. The solution was concentrated to dryness to give 0.112 g (100% yield) of sodium salt 11. Compound 11 is soluble in water and ¹H NMR is similar to that of 1.

2,2′-Bis-[(4-hydroxy-sodio-aminocarbonyl)phenoxy]ethyl ether (12). By a procedure similar to that above described for the preparation of 11, 0.10 g of 3 gave 0.112 g (100% yield) of 12. Compound 12 is soluble in water and the ¹H NMR spectrum is similar to that of 3.

### Example II

### Inhibitory Activities of Example 1 Compounds Against Plasmodium and Leishmania

The analogous methyl esters, 5, 7, and 9, and carboxylic acids, 6, 8, and 10, were used to test whether the bishydroxamic acid function is important for the bioactivities. The sodium salts of 1 and 3, compounds 11 and 12, are soluble in water and were also evaluated for their biological activities. The Table summarizes data of the inhibition of P. falciparum D6 and W2 intraerythrocytic forms and L. donovani axenic amastigote-like parasites. While bisesters 5, 7, and 9, and biscarboxylic acids 6, 8, and 10 do not show inhibitory activities, hydroxamic acids 1-3 show significant inhibitory activities. Hence, IC₅₀ values of hydroxamic acids 1, 2, and 3 are 2.2, 0.69, and 0.24 μM, respectively, for D6, and 3.2, 1.05, and 0.35 μM, respectively, for W2. Bishydroxamic acid sodium salt 12 exhibits the strongest activities, with IC₅₀ values of 0.26 μM for D6 and 0.36 μM for W2. Chloroquine and mefloquine were used for comparison, their IC₅₀ values being 0.013 μM (both) for D6, and 0.45 μM and 8 nM, respectively, for W2. Moreover, bishydroxamic acid 3 and its sodium salt 12 inhibit L. donovani parasites with IC₅₀ values of 18.5±6.0 and 37.4±14.0 μM, respectively, with pentamidine used for comparison. It appears that the inhibitory activities against P. falciparum D6 and W2 and L. donovani parasites of bishydroxamic acid 3 are similar to its sodium salt 12. The compounds are more active in vitro against Plasmodium, perhaps due to the vigorous metabolism of hemoglobin by the parasite. Compound 12 is soluble in water, while compound 3 is soluble in DMSO but only slightly soluble in water. The results demonstrate that bishydroxamic acid functionality is essential for the inhibition of malaria parasites. It is possible that the bishydroxamic acids inhibit hypoxanthine-guanine phosphoribosyltransferases, [15] which lead to antiparasitic activities. The simplicity of the molecular structures of bishydroxamic acids described above may provide a new entry in providing potentially active antimalarial agents that may be used on chloroquine and mefloquine resistant parasites.

TABLE
In vitro inhibitory activities against P. Falciparum
D6 and W2 and L. Donovani parasites

| | | | | W2, IC₉₀ | L. Donovani |
|---|---|---|---|---|---|
| Compounds | D6, IC₅₀ (μM) | D6, IC₉₀ (μM) | W2, IC₅₀ (μM) | (μM) | parasites (μM) |
| Bisester 5 | >60 | NT | >60 | NT | NT |
| Bisester 7 | >60 | NT | >60 | NT | NT |
| Bisester 9 | >60 | NT | >60 | NT | >100 |
| Biscarboxylic acid 6 | >60 | NT | >60 | NT | NT |
| Biscarboxylic acid 8 | >60 | NT | >60 | NT | NT |
| Biscarboxylic acid 10 | >38 | NT | >38 | NT | >100 |
| Bishydroxamic acid 1 | 2.2 | 6.2 | 3.2 | NT | NT |
| Bishydroxamic acid 2 | 0.69 | 1.8 | 1.05 | 2.57 | NT |
| Bishydroxamic acid 3 | 0.24 | 0.47 | 0.35 | 0.60 | 18.5 ± 6.0 |
| Bishydroxamic acid sodium | 1.0 | 2.0 | 1.09 | NT | NT |
| salt 11 | | | | | |
| Bishydroxamic acid sodium | 0.26 | 0.52 | 0.36 | 0.60 | 37.4 ± 14.0 |
| salt 12 | | | | | |
| Chloroquine | 0.013 | 0.014 | 0.45 | 0.65 | NT |
| Mefloquine | 0.013 | 0.24 | 8 nM | 0.015 | NT |
| Pentamidine | NT | NT | NT | NT | 1.19 ± 0.12 |

NT, not tested

The following sets forth the test protocols used to generate the date in the foregoing table.

Inhibition of the growth of P. falciparum. A reported method [16] for the inhibition of the growth of P. falciparum was followed. The incubation period of the parasites was 66 h and the starting parasitemia was 0.2% with a 1% hematocrit. The medium used was RPMI-1640 culture medium with no folate or p-aminobenzoic acid and 10% normal heat-inactivated human plasma, and P. falciparum D6 and W2 clones were used. D6 is a clone from the Sierra I/UNC isolates and is susceptible to chloroquine and pyrimethamine but has reduced susceptibilities to mefloquine and halofantrine. W2 is a clone of the Indochina I isolate and is resistant to chloroquine and pyrimethamine but susceptible to mefloquine. Compounds were dissolved in DMSO, diluted 400-fold with complete culture media, and then diluted 2-fold, 11 times, to give a concentration range of 1048-fold by a Biomek 1000 or 2000 liquid handling system into 96-well microtiter plates. The diluted compounds were transferred (25 L) to test plates, 200 L of parasitized erythrocytes (0.2% parasitemia and 1% hematocrit) was added, and was incubated at 37° C. in a controlled environment of 5% CO₂, 5% O₂, and 90% N2. After 42 h, 25 L of [³H]-hypoxanthine was added and the plates were incubated for an additional 24 h. The plates were then frozen at −70° C. to lyse the red cells and later thawed and harvested onto glass fiber filter mats by using a 96-well cell harvester. The filter mats were counted in a scintillation counter and the data were downloaded with the custom, automated analysis software developed by WRAIR. For each compound, the concentration-response profile was determined and 50% inhibitory concentrations (IC₅₀) were determined by using a nonlinear, logistic dose-response analysis program.

Inhibition of the growth of L. donovani parasites. A reported method [17] for the inhibition of the growth of L. donovani parasites was followed. L. donovani axenic amastigotes were maintained in modified RPMI medium as previously described. [17] Inhibition of growth by the compounds was measured in a 3-day assay using the tetrazolium dye-based CellTiter 96 Aqueous Non-Radioactive Cell Proliferation Assay Kit (Promega, Madison, Wis., USA) as previously described. [17] The known anti-leishmaniasis drug, pentamidine, was used as a control.

### Example III

### Metal Binding Affinities of Bishydroxamic Acids

In order to determine the binding affinity of bishydroxamic acids (such as 2, 3, 15, and 16) with various metal ions such as zinc, iron, copper, and magnesium, various spectroscopic methods are used. First, competitive binding experiments of various bishydroxamic acids with different metal ions are studied. In brief, a known concentration of a bishydroxamic acid is treated with different known concentrations of metal salts separately in aqueous solution. The concentrations of the metal bound bishydroxamic acids are determined using UV-visible spectrometry and HPLC-MALDI mass spectrometry. Varying the concentrations of metal salts provide different concentrations of the bound metal bishydroxamic acids and the concentrations are quantified. Second, a spectrophotometric determination is performed to study the binding of bishydroxamic acids with different metal ions. After treatment of bishydroxamic acids with metal ions, a diazo dye, 4-(2′-pyridylazo)resorcinol, is added to chelate with the unbound metal ions. Concentrations of the azo bound metal species are quantified by UV-vis spectrometry. From this study, the binding selectivity of various bishydroxamic acids with different metal ions are determined. The most selective zinc chelator is the best candidate for ejecting zinc from zinc-finger motifs.

### Example IV

### Biological Evaluation of Zinc Ejection

The biological evaluation is divided into the following sequence of procedures:

(i) Using automated peptide synthesizer, zinc finger I KCFNCGKEGHTARNCR (Seq ID No.1), zinc finger II GCWKCGKEGHQMKDCT (Seq ID No.2), and the combination of two zinc fingers KCFNCGKEGHTARNCR-APRKK-GCWKCGKEGHQMKDCT-ERQAN (Seq ID No. 3) are prepared.

(ii) Zinc (2+) ion is inserted into these two zinc-finger peptides.

(iii) At physiological pH (7.4), the ejection of zinc ions from the above zinc fingers with the above bishydroxamic acids (compounds 1-3, 15A-15D, and 16) is studied. In brief, an experiment is carried out as follows. Zinc containing peptide (such as zinc finger I) is treated with bishydroxamic acid in pH 7.4. Zinc binding peptides (ca M.W.=1800, 4000 Da or greater) and zinc-chelating bishydroxamic acid (less than 1000 Da) are separated using Slide-A-Lyzer Dialysis Cassettes (Pierce Biotechnology, Inc., Rockford, Ill.) or Sephadex G-10 gel filtration resins (Sigma., St. Louis, Mo.) in de-salted spin column. The small molecules (such as bishydroxamic acid) are trapped and the peptide (with or without zinc) is present in the filtrate. The concentration will be quantified by HPLC-UV mass spectrometry.

(iv) Alternatively, the zinc ejection experiment is conducted using Surface Plasmon Resonance (SPR) spectroscopy (a BIAcore 3000; Pharmacia Biosensor AB. The zinc finger peptide is attached to the gold CM5 chip of SPR by activating the carboxylic acid function of the Sensor chip CM5 with N-hydroxysuccinimide followed by attaching the peptide on the amino terminus. The concentration of the bound peptide onto CM5 chip is determined. Zinc ion in a pH 7.4 buffer solution is passing through the peptide bound CM5 chip. Again, the concentration of the zinc bound peptide is determined from the response unit of SPR. Next, bishydroxamic acid is passing through the zinc-peptide bound CM5 chip. If the zinc is ejected from the peptide and bound to bishydroxamic acid, after passing pH 7.4 buffer to remove all bishydroxamic acid, the SPR response unit changes and the amount of ejected zinc is quantified. Hence, by using this method, different metals are tested and the binding affinity of bishydroxamic acids are determined.

Although normal cells also contain zinc-finger proteins, however, infected cells usually grow at faster rates and have preferential uptake of nutrients, metals, drugs etc. Since infected subjects will only be treated acutely and for a short time. The normal cells in the subject will most likely recover.

Recent studies indicate that the condensation of various molecules and proteins with the 14 amino acid C-terminal sequence from the human immunodeficiency virus type-1 GRKKRRQRRRPPQG (Seq ID No.4), leads to nuclear targeting and translocation. The Tat-sequence contains the RGD motif, which is important in binding to integrins and subsequent cellular uptake. This approach could be used with the compounds hereof. For example, consider the compound depicted in following Figure. By specifically targeting the compound to the nuclear membrane reduced concentrations might show acceptable efficacy and thereby reduce potential negative side-effects. It should be noted that Tat-conjugated proteins had been reported (Watanabe, N.; Iwamoto, T.; Bowen, K. D.; Dickinson, D. A.; Torres, M.; Forman, H. J. Bio-effectiveness of Tat-catalase conjugate: a potential tool for the identification of H2O2-dependent cellular signal transduction pathways. Biochem. Biophys. Res. Commun. 2003, 303, 287-293.). The compound of the following figure can be synthesized from compound 15D by the sequence: (i) activation of the carboxyl terminus of human immunodeficiency virus type-1 with N-hydroxysuccinimide; and (ii) coupling of this activated peptide with 15D.

Thus, the present invention provides a convenient method to search for potent and selective antiviral drugs that possess inhibitory activity against replication of viral RNA.

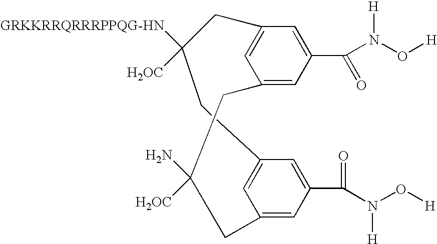

### REFERENCES

1. Zhang, P.; Nicholson, D. E.; Bujnicki, J. M.; Su, X,; Brendle, J. J.; Fedig, M.; Kyle, D. E.; Milhous, W. K.; Chiang, P. K. J. Biomed. Sci. 9 (2002), p. 34.
2. Bhattacharya, G.; Salem, M.; Werbovetz, K. A. A Bioorg. Med. Chem. Lett. 12 (2002), p. 2395.
3. Andrew, K. T.; Walduck, A.; Kelso, M. J.; Fairlie, D. P.; Saul, A.; Parsons, P. G. Int. J. Parasitol. 30 (2000), p. 761.
4. (a) Inhibitors of histone deacetylases: R. Breslow, B. Jursic, Z. F. Yan, E. Friedman, L. Leng, L. Ngo, R. A. Rifkind and P. A. Marks. Proc. Natl. Acad. Sci. USA. 88 (1991), p. 5542. (b) M. Richon, S. Emiliani, E. Verdin, Y. Webb, R. Breslow, R.; Rifkind, R. A.; and P. A. Marks. Proc. Natl. Acad. Sci. U.S.A. 95 (1998), p. 3003. (c) R. D. Glick, S. L. Swendeman, D. C. Coffey, R. A. Rifkind, P. A. Marks, V. M. Richon and M. P. La Quaglia. Cancer Res. 59 (1999), p. 4392. (d) 5-Lipoxygenase inhibitors: K. A. Ohemeng, V. N. Nguyen, C. F. Schwender, M. Singer, M. Steber, J. Ansell and W. Hageman. Bioorg. Med. Chem. 2 (1994), p. 187.
5. P. Desaraju and A. Winston. J. Coord. Chem. 14 (1986), p. 241.
6. M. Heitzer and A. Hallmann. J. Biol. Chem. 277 (2002), p. 28280.
7. C. Voyiatzaki and K. Soteriadou. J. Biol. Chem. 267 (1992), p. 9112.
8. K. P. Soteriadou, P. Papavassiliou, C. Voyiatzaki and J. Boelaert. J. Antimicrob. Chemother. 35 (1995), p. 23.
9. A. S. Reddy, M. S. Kumar and G. R. Reddy. Tetrahedron Lett. 41 (2000), p. 6285.
10. Baettig, K.; Gerald, C. J. Photographic silver-dye-bleach material containing azo dye. Eur. Pat. Appl. EP 89-312507, 1990.
11. Terada, H.; Ishii, M.; Tokuda, K.; Imaida, T.; Takashima, S. Dyeable polyesters. Jpn. Pat. Appl. JP 42016073, 1967.
12. (a) A number of publications and patents have appeared and only the original first and latest reports are listed here: A. Conix. J. Polym. Sci. 29 (1958), p. 343. M. Mizuno, A. Hirai, H. Matsuzawa, K. Endo, M. Suhara, M. Kenmotsu and C. D. Han. Macromolecules 35 (2002), p. 2595.
13. G. S. Peters, S. D. Gorham, F. J. Smith, A. M. Fraser, M. E. Colclough and R. Millar. Propellants, Explosives, Pyrotechnics 25 (2000), p. 191.
14. A representative publication: W. X. Zhang and F. Higashi. J. Polym. Sci., Part A: Polymer Chem. 31 (1993), p. 2935.
15. (a) C. M. Li, P. C. Tyler, R. H. Furneaux, G. Kicska, Y. Xu, C. Grubmeyer, M. E. Girvin and V. L. Schramm. Nat. Struct. Biol. 6 (1999), p. 582. (b) W. Shi, C. M. Li, P. C. Tyler, R. H. Furneaux, C. Grubmeyer, V. L. Schramm and S. C. Almo. Nat. Struct. Biol. 6 (1999), p. 588.
16. (a) D. Opsenica, G. Pocsfalvi, Z. Jurnaic, B. Tinant, J.-P. Declerq, D. E. Kyle, W. K. Milhous and B. A. Solaja. J. Med. Chem. 43 (2000), p. 3274. (b) R. E. Desjardins, C. J. Canfield, D. E. Haynes and J. D. Chulay. Antimicrob. Agents Chemother. 16 (1979), p. 710.
17. K. A. Werbovetz, J. J. Brendle and D. L. Sackett. Mol. Biochem. Parasitol. 98 (1999), p. 53.

1. A method chelating zinc from a zinc finger peptide comprising the step of contacting said peptide with an effective amount of a bishydroxamic acid or salt thereof.

2. The method of claim 1, said acid being a symmetrical bishydroxamic acid.

3. The method of claim 1, said acid selected from the group consisting of compounds 2, 3, 15A-15D, inclusive, and 16.

4. The method of claim 1, said method being in vitro.

5. The method of claim 1, said method being in vivo.

6. A method of viral inhibition comprising the step of contacting a target with an effective amount of a bishydroxamic acid or salt thereof.

7. The method of claim 6, said acid being a symmetrical bishydroxamic acid.

8. The method of claim 6, said acid selected from the group consisting of compounds 2, 3, 15A-15D, inclusive, and 16.

9. The method of claim 6, said method being in vitro.

10. The method of claim 6, said method being in vivo.

11. A method ejecting zinc from a zinc finger peptide comprising the step of contacting said peptide with an effective amount of a bishydroxamic acid or salt thereof.

12. The method of claim 11, said acid being a symmetrical bishydroxamic acid.

13. The method of claim 11, said acid selected from the group consisting of compounds 2, 3, 15A-15D, inclusive, and 16.

14. The method of claim 11, said method being in vitro.

15. The method of claim 11, said method being in vivo

16. A compound selected from the group consisting of compounds 2, 3, 15, and 16.

17. A method of malarial inhibition comprising the step of contacting a malarial parasite with an effective amount of bishydroxamic acid or salt thereof.

18. The method of claim 17, said acid being a symmetrical bishydroxamic acid.

19. The method of claim 17, said acid selected from the group consisting of compounds 2, 3, 15A-15D, inclusive, and 16.

20. The method of claim 17, said method being in vitro.

21. The method of claim 17, said method being in vivo.


SEQUENCE LISTING (4 sequences)

SEQ ID NO: 1 - protein, 16 aa, Human immunodeficiency virus type 1
KCFNCGKEGHTARNCR

SEQ ID NO: 2 - protein, 16 aa, Human immunodeficiency virus type 1
GCWKCGKEGHQMKDCT

SEQ ID NO: 3 - protein, 42 aa, Human immunodeficiency virus type 1
KCFNCGKEGHTARNCRAPRKKGCWKCGKEGHQMKDCTERQAN

SEQ ID NO: 4 - protein, 14 aa, Human immunodeficiency virus type 1
GRKKRRQRRRPPQG